(19) United States
(12) Patent Application Publication
(10) Pub. No.: US 2026/0297540 A1
(43) Pub. Date: Oct. 1, 2026

(54) CELL PRESERVATION SOLUTION AND CELL PRESERVATION METHOD

(71) Applicant: UNIDT (Shanghai) Co., Ltd., Shanghai (CN)

(72) Inventor: Wei Tang, Monmouth Junction, NJ (US)

(73) Assignee: UNIDT (Shanghai) Co., Ltd., Shanghai (CN)

(21) Appl. No.: 18/996,454

(22) PCT Filed: Sep. 21, 2022
(86) PCT No.: PCT/CN2022/120197

§ 371 Date: Jan. 17, 2025

(30) Foreign Application Priority Data
Aug. 2, 2022 (CN) 202210922177.1

(51) Int. Cl. C12N 5/00

(52) U.S. Cl. CPC C12N 5/522

(57) ABSTRACT

One aspect of the embodiments relates to a cell preservation solution, comprising: 20 mM~50 mM of sucrose; 20 mM~50 mM of mannitol, 10 mM~50 mM of lactobionic acid, 5 mM~20 mM of glucose, 1 mM~3 mM of adenosine, 1 mM~5 mM of reduced glutathione, 1 mM~2 mM of dextran-40, 10 mM~30 mM of potassium dihydrogen phosphate, 10 mM~30 mM of potassium carbonate, 10 mM~30 mM of potassium chloride, 5 mM~30 mM of sodium chloride, 10 mM~60 mM of sodium hydroxide, 10 mM~60 mM of potassium hydroxide, 0 mM~20 mM of vitamin E, 0 mM~25 mM of 4-(2-hydroxyethyl)piperazine-1-ethanesulfonic acid, and 0%~10% dimethyl sulfoxide. Another aspect of the embodiments relates to a cell preservation method, which uses the cell preservation solutions described in the present application to store cells.

The present application claims priority to Chinese Patent Application No. 202210922177.1, filed with the China National Intellectual Property Administration (CNIPA) on Aug. 2, 2022, with the invention title “cell preservation solution, cell preservation method,” the content of which is incorporated by reference in its entirety into the present application.

### TECHNICAL FIELD

The present invention relates to the field of modern regenerative medicine, in particular, to a cell preservation solution and cell preservation method.

### BACKGROUND TECHNOLOGY

Modern regenerative medicine, such as gene therapy, tumor cell therapy, novel antibody therapy, stem cell regeneration therapy, etc., is an emerging medical therapy field, which is characterized by the transformation of isolated living cells to make therapeutic cells, which are then infused/transplanted into a patient to achieve the treatment goal. Generally speaking, isolated living cells must be preserved by refrigerating and freezing at low temperature to ensure that the viability and functionality of the cells are maintained during storage and transportation. In other words, cell preservation technology through low temperature refrigeration, and freezing (cryopreservation technology) can be described as an indispensable part of modern regenerative medicine.

The development of cell low temperature refrigeration, freezing cryopreservation technology needs to be based on the thorough research and understanding of cell cryobiology. In order to effectively store cells refrigerated, frozen at low temperature, it is necessary to avoid the damage to cells by low temperature refrigeration and freezing to the greatest extent according to the biological characteristics of cell cryogenesis and maintain cell viability and functionality. Specifically, at normal physiological temperatures, the intracellular fluid and extracellular fluid are in an isothermal and isotonic state, and the cells are metabolically active, which requires the consumption of oxygen and nutrients, and at the same time removal of a large amount of free radicals and peroxides produced to maintain a normal physiological state. However, in the low-temperature refrigerated and frozen state, cells may experience various degrees of damage, affecting normal physiological activities, and even death. The damage caused by low temperature refrigeration and freezing to cells may be multifaceted, such as osmotic damage, mechanical damage, chemical damage, and so on. For example, in the cooling process of refrigeration and freezing, ice crystals may first form in the extracellular interstitial environment, the interstitial fluid is concentrated, and the osmotic pressure increase rises, resulting in the intracellular water seeping out through the cell membrane, and the electrolyte concentration inside the cell increases, resulting in pH changes, causing protein denaturation, lysosomal destruction, loss of membrane protein function, membrane leakage rupture, etc., and ultimately leading to cell death. At the same time, further cooling may produce ice crystals inside the cell, which may pierce and squeeze the cell membrane and organelles, resulting in mechanical damage to the cell membrane and death. On the other hand, a large amount of free radicals and peroxides inside cells can also induce apoptosis in low-temperature refrigerated and frozen environments.

The existing cell preservation solutions and cell preservation methods can prevent the damage to cells by low temperature refrigeration and freezing to a certain extent, but there are still shortcomings. For example, when the existing cell preservation solutions and cell preservation methods are applied in clinical practice, there may be a risk of adverse effects to the human body, there may be a risk of intracellular deoxyribonucleic acid (DNA) distortion and protein denaturation, and the possibility that the human body may have a non-compatible response to the preserved cells. In addition, some of the components of existing cell preservation solutions not only are source limited and expensive, but also carry the potential risk of contamination by other microorganisms such as viruses, as well as possibly cause side effects, such as allergies, etc., to the human body.

### Content of the Invention

The object of the present invention is to provide improved cell preservation solutions and cell preservation methods.

In one aspect, the embodiment of the present invention relates to one type of cell preservation solution, which comprises: 20 mM~50 mM sucrose; 20 mM~50 mM mannitol; 10 mM~50 mM lactobionic acid; 5 mM~20 mM glucose; 1 mM~3 mM adenosine; 1 mM~5 mM reduced glutathione; 1 mM~2 mM dextran-40; 10 mM~30 mM potassium dihydrogen phosphate; 10 mM~30 mM potassium carbonate; 10 mM~30 mM potassium chloride; 5 mM~30 mM sodium chloride; 10 mM~60 mM sodium hydroxide; 10 mM~60 mM Potassium hydroxide; 0 mM~20 mM vitamin E; 0 mM~25 mM 4-hydroxyethylpiperazineethanesulfonic acid; and 0%~10% dimethyl sulfoxide.

In some embodiments, the cell preservation solution comprises 0.5 mM~20 mM vitamin E and 0% dimethyl sulfoxide.

In some embodiments, the cell preservation solution comprises 2 mM~5 mM vitamin E.

In some embodiments, the cell preservation solution comprises 0 mM~20 mM 4-hydroxyethylpiperazine ethanesulfonic acid.

In some embodiments, the cell preservation solution comprises 0 mM vitamin E and >0% dimethyl sulfoxide.

In some embodiments, the cell preservation solution comprises 0 mM~20 mM 4-hydroxyethylpiperazine ethanesulfonic acid.

In some embodiments, the cell preservation solution comprises 5%~10% dimethyl sulfoxide. In some embodiments, the pH value of the cell preservation solution is in the range of 7.0~8.0.

In some embodiments, the osmotic pressure of the cell preservation solution is 300~400 milliosmoles (mOsm).

In some embodiments, the cell preservation solution comprises water.

In another aspect, the present invention relates to a type of cell preservation method, wherein cells are preserved with cell preservation solutions described in the present application.

In some embodiments, the cell preservation method preserves the cells at 2° C.~8° C. or −80° C.~−196° C.

In some embodiments, the cells comprise one or more types of stem cells, immune cells, and tumor cells.

In some embodiments, the cells comprise human colon cancer cells.

When the technical conditions permit, the technical schemes of each embodiment in the present application may be arbitrarily combined.

The present application is further described below in conjunction with the accompanying drawings.

### DESCRIPTION OF THE ACCOMPANYING DRAWINGS

FIG. 1 shows the experimental data of cell metabolic activity obtained in Example 5.

FIG. 2 shows the experimental data of cell proliferation activity obtained in Example 5.

FIG. 3 shows the experimental data of cell metabolic activity obtained in Example 10.

FIG. 4 shows the experimental data of cell proliferation activity obtained in Example 10.

### DESCRIPTION OF EMBODIMENTS

The present invention is further described below in conjunction with specific embodiments. It should be understood that these embodiments are intended only to illustrate the invention and are not intended to limit the scope of protection of the invention. In practical application, the improvements and adjustments made by those skilled in the art according to the present invention still belong to the scope of protection of the present invention.

In one aspect, the embodiment of the present invention relates to a cell preservation solution, which comprises: 20 mM~50 mM sucrose; 20 mM~50 mM mannitol; 10 mM~50 mM lactobionic acid; 5 mM~20 mM glucose; 1 mM~3 mM adenosine; 1 mM~5 mM reduced glutathione; 1 mM~2 mM dextran-40; 10 mM~30 mM potassium dihydrogen phosphate; 10 mM~30 mM potassium carbonate; 10 mM~30 mM potassium chloride; 5 mM~30 mM sodium chloride; 10 mM~60 mM sodium hydroxide; 10 mM~60 mM Potassium hydroxide; 0 mM~20 mM vitamin E; 0 mM~25 mM 4-hydroxyethylpiperazine ethanesulfonic acid; and 0%~10% dimethyl sulfoxide.

The cell preservation solutions in the embodiments of the present invention have defined compositions, stable performance, are safe and reliable, and can be used directly in the human body. The cell preservation solutions in the embodiments of the present invention have a relatively low, or 0, amount of dimethylsulfoxide (DMSO), and when applied to clinical practice, unlikely to have the risk of producing adverse effects to the human body, and unlikely to cause intracellular deoxyribonucleic acid (DNA) distortion and protein denaturation, and the possibility of the human body's incompatibility to cells they preserved is small. In addition, the components of the cell preservation solutions in the embodiments of the present invention have relatively wide sources, low prices, and do not contain exogenous animal serums, exogenous proteins, etc., and are unlikely to have the potential risk of contamination by other microorganisms, such as viruses, and are unlikely to cause side effects such as allergy, etc., to the human body.

In the embodiments of the present invention, unless otherwise specifically indicated, the numerical value may contain errors including calculation errors, precision errors, measurement errors, etc., such as error in the range of plus or minus 5%. For example, 10% can include values in the range of 10%×(1±5%), i.e. values in the range of 9.5% to 10.5%.

In the embodiments of the present invention, unless otherwise specifically indicated, the numerical range may include any sub-range therein, for example, 0%~10% may include 5%~10%, 0%~5%, 3%~8%, and so on.

The sucrose can serve as a non-permeable extracellular cryoprotectant, protecting the cell membrane from cold shock. Said sucrose can also serve as an energy source for cellular metabolism during cell storage.

The mannitol can serve as an extracellular non-permeable substance, improving extracellular osmotic pressure, and can also serve as an effective hydroxide free radical scavenger.

The lactobionic acid can serve as a non-permeable anion that can be used to counteract cell swelling during low temperatures. The lactobionic acid can be biocompatible and can be a strong chelating agent of calcium and iron, which can help reduce cell damage due to calcium influx and free radical formation.

The glucose can serve as the main source of energy for cell metabolism in the process of cell storage, through the anaerobic digestion pathway or aerobic and tricarboxylic acid cycle in the mitochondria to generate carbon dioxide and water, release adenosine triphosphate (Adenosine triphosphate, ATP), provide energy for cellular life activities.

The adenosine can serve as a substrate for the regeneration of adenosine triphosphate (ATP) in the mitochondria of the cell, which can be involved in providing energy for cellular life activities.

The reduced glutathione can serve as an important antioxidant and hydroxyl radical scavenger in the cell, protect the sulfhydryl groups in the molecules such as intracellular proteins and enzymes, and protect the cell membrane from the damage by free radicals. The reduced glutathione can also serve as a cofactor of glutathione peroxidase, which can promote the metabolism of lipid peroxide and hydrogen peroxide.

The dextran-40 can serve as a non-permeable extracellular cryoprotectant, which can, at low temperatures, counteract the colloidal osmotic pressure produced by negatively charged proteins and other metabolites inside the cell, thus preventing the cell from cracking due to water absorption-induced expansion.

The vitamin E can serve as an antioxidant that can scavenge intracellular oxygen free radicals at low temperatures, thereby preventing damage to cell membranes by free radicals. Said vitamin E can also inhibit the release of nitric oxide. The vitamin E can also protect cells from freezing-induced apoptosis.

The 4-hydroxyethylpiperazine-N-2-ethanesulfonic acid (HEPES) can serve as a large sulfonic acid biological pH solution, which can have excellent buffering capacity at low temperatures, prevent acidosis, and will not penetrate into cells, helping to prevent osmotic swelling.

The dimethyl sulfoxide (DMSO) can serve as a permeable cell protective agent, which can quickly penetrate the cell membrane into the cell, lower the freezing point, delay the cryopreservation process, and at the same time increase the intracellular ion concentration, reduce the formation of intracellular ice crystals, thereby reduce cell damage. The corresponding amount and concentration percentage of the dimethyl sulfoxide can be the volume percentage, i.e., 0%~10% can represent the ml amount of the dimethyl sulfoxide in 100 ml of the said cell preservation solution is 0~10.

The potassium dihydrogen phosphate, the potassium carbonate, the potassium chloride, the sodium chloride, the sodium hydroxide, and the potassium hydroxide may be electrolytes, respectively, useful to maintain the intracellular pH level at low temperature and the cellular sodium potassium ion pump.

The cell preservation solutions in the embodiments of this application can be used to refrigerate cells at low temperatures such as 2° C.~8° C. for a short period to maintain cell viability and functionality, or freeze cells at deep low temperatures such as −80° C.~−196° C. for a long period to maintain cell viability and functionality.

In some embodiments, the cell preservation solutions comprise 0.5 mM~20 mM vitamin E and 0% dimethyl sulfoxide.

In this way, it can help the cell preservation solutions to maintain cell viability and functionality during the process of cold storage of cells at low temperature such as 2° C.~8° C. for up to 5 days, 7 days and beyond. Moreover, the content of dimethylsulfoxide (DMSO) in the cell preservation solutions is 0, and when applied to clinical practice, it is unlikely to have the risk of producing adverse effects to the human body, and it is unlikely to cause intracellular deoxyribonucleic acid (DNA) aberration and protein denaturation, and the possibility of the human body's incompatibility to the cells it preserved is relatively small.

In some embodiments, the cell preservation solution comprises 2 mM~5 mM vitamin E.

In this way, it can help the cell preservation solutions to scavenge intracellular oxygen free radicals when the cells are stored in low temperature refrigeration at such as 2° C.~8° C., thereby preventing the damage of free radicals to the cell membrane, inhibiting the release of nitric oxide, and protecting the cells from freezing-induced apoptosis.

In some embodiments, the cell preservation solutions comprise 0 mM~20 mM 4-hydroxyethylpiperazine ethanesulfonic acid.

In this way, it can help the cell preservation solutions to have excellent buffering capacity when the cells are stored at low temperature refrigeration such as 2° C.~8° C., preventing acidosis, and will not penetrate into the cells, helping to prevent osmotic swelling.

In some embodiments, the cell preservation solutions comprise 0 mM vitamin E and >0% dimethyl sulfoxide.

In this way, it can help the cell preservation solutions to quickly penetrate the cell membrane into the cell in the process of cryopreservation of cells at deep low temperature, such as −80° C.~−196° C., as long as, for example, months, years, decades, and longer, so as to reduce the freezing point, delay the cryopreservation process, and at the same time increase the intracellular ion concentration, reduce the formation of intracellular ice crystals, thereby reduce cell damage, and maintain cell viability and functionality.

In some embodiments, the cell preservation solutions comprise 0 mM~20 mM 4-hydroxyethylpiperazine ethanesulfonic acid.

In this way, when cryopreservation of cells at deep low temperature such as −80° C.~−196° C., it can help the cell preservation solutions to have excellent buffering capacity to prevent acidosis, and not penetrate into the cells, helping to prevent osmotic swelling.

In some embodiments, the cell preservation solutions comprise 5%~10% dimethyl sulfoxide.

In this way, the cell preservation solutions can, when the cells are cryopreserved at such as −80° C.~−196° C., quickly penetrate the cell membrane into the cell, reduce the freezing point, delay the cryopreservation process, at the same time, increase the ion concentrations in the cells reduce the formation of ice crystals in the cells, thereby reducing the cell damage.

In some embodiments, the pH value of the cell preservation solution is in the range of 7.0~8.0.

In this way, it can help the cell preservation solutions to provide a physiological environment suitable for the cells.

In some embodiments, the osmotic pressure of the cell preservation solutions is 300~400 milliosmoles (mOsm).

In this way, it can help the cell preservation solutions to provide a physiological environment suitable for the cells. The osmotic pressure can be the sum of the moles of the individual components in the cell preservation solution.

In some embodiments, the cell preservation solutions comprise water.

In this way, it can help the cell preservation solutions to provide a physiological environment suitable for the cell. Components other than the water of the cell preservation solutions can form a mixture in the water.

On the other hand, an embodiment of the present invention relates to a cell preservation method wherein cells are preserved with cell preservation solutions described in the present application.

The cell preservation solutions used in the cell preservation method in the embodiments of the present invention have defined compositions, stable performance, are safe and reliable, and can be directly used in the human body. The amount of dimethylsulfoxide (DMSO) in the said cell preservation solutions used in the said cell preservation method in the embodiments of the present invention is relatively low, or is 0, when used in clinical practice, it is unlikely to have the risk of produce adverse effects to the human body, it is unlikely to cause intracellular deoxyribonucleic acid (DNA) aberration and protein denaturation, and the possibility of the human body's incompatibility to the cells it preserved is relatively low. In addition, the components of the cell preservation solutions used in the cell preservation method in the embodiments of the present invention have wide sources, low prices, and do not contain exogenous animal serum, are unlikely to have the potential risk of contamination by other microorganisms, such as viruses, and are unlikely to cause side effects such as allergy, etc., to the human body.

In some embodiments, the cell preservation method preserves the cells at 2° C.~8° C. or −80° C.~−196° C.

In this way, it can help to ensure that the cell viability and functionality are maintained during storage and transportation process for a short period of time, or for a long time. For example, the cells can be refrigeration preserved at 2° C.~8° C. for 5 days, 7 days, or longer with the cell preservation method. Alternatively, the cells may be freezing preserved at −80° C.~−196° C. for months, years, decades, or longer using the cell preservation method.

In some embodiments, the cell preservation method may comprise that after the cells to be preserved are collected by centrifugation, the cells are resuspended with the cell preservation solution, so that the cell concentration reaches for example, 2×10⁶/ml~1×10⁷/ml, and is stored in a refrigerator at 2° C.~8° C. for a period of up to 5 days, 7 days or longer.

In some embodiments, the cell preservation method may comprise that the cells to be preserved are cultured adherently to the required number in a petri dish, the cell culture medium is aspirated, the cell preservation solution is added, and the cells are stored in a refrigerator at 2° C.~8° C. for a period of up to 5 days, 7 days or longer.

In some embodiments, the cell preservation method may comprise that after the cells to be preserved are collected by centrifugation, the cells are resuspended with the cell preservation solution, so that the cell concentration reaches 2×10⁶/ml~1×10⁷/ml, moved into a cryovial, placed in a condition at −80° C. temperature to cool down at a cooling rate of about 1° C./min, and after overnight, moved into liquid nitrogen (−196° C.) for long-term storage. It can be stored for decades.

In some embodiments, the cells comprise one or more types of stem cells, immune cells, and tumor cells.

In this way, it can help to ensure that one or more types of the stem cells, immune cells, and tumor cells maintain viability and functionality during storage and transportation.

In some embodiments, the cells include human colon cancer cells.

In this way, it can help to ensure that the human colon cancer cells maintain viability and functionality during storage and transportation.

The rewarming method for the preserved cells refrigerated and frozen in the said cell preservation solutions by the said cell preservation method in the embodiment of the present application may comprise: after the cells of the refrigeration and freezing preserved are collected by centrifugation, the cell preservation solution is aspirated, the cells are resuspended with the cell culture medium, and continued to be cultured.

The functionality measurement method of the preserved cells refrigerated and frozen in the said cell preservation solutions by the said cell preservation method in the embodiment of the present application may comprise: after the preserved cells are rewarmed and cultured for 1 day, the cell metabolic activity is measured with cell viability detection reagent, such as ALAMAR BLUE™ Within 1 day after cell rewarming, there may be lag apoptosis, and the cells tend to stabilize after 1 day, so the measurement after 1 day can more truly reflect cell viability.

When the preserved cells refrigerated and frozen in the said cell preservation solution by the said cell preservation method in the embodiments of the present application need to be used in clinical practice, they can be directly reinfused into the body after resuspension.

The experimental Examples in the present application are intended primarily to aid in understanding the embodiments of the invention and are not intended to constitute a limitation on the scope of the claims. Referring to the following experimental Examples, it can be seen that the cell preservation solutions of the embodiment of the present application have simple formula, relative low cost, and comparable to or even better performance than that of commercially available products.

### Examples 1-4

Per the compositions and concentrations of the cell preservation solutions corresponding to the experimental Examples 1~4 shown in Table 1 below, individually weighed sucrose, mannitol, lactobionic acid, glucose, adenosine, reduced glutathione, dextran-40, potassium dihydrogen phosphate, potassium carbonate, potassium chloride, sodium chloride, potassium hydroxide, vitamin E, 4-(2-Hydroxyethyl)piperazine-1-ethanesulfonic acid (HEPES), prepared 4 kinds of mixed solutions with purified water, titrated pH of each to 7.6 with sodium hydroxide, and filtered through a 0.2 μm sterile filter membrane, and the filtered filtrates were the 4 kinds of cell preservation solutions in experimental Examples 1-4.

TABLE 1

| | Concentration | | | |
|---|---|---|---|---|
| | Example | Example | Example | Example |
| Ingredient | 1 | 2 | 3 | 4 |
| Sucrose, mM | 40 | 40 | 40 | 40 |
| Mannitol, mM | 40 | 40 | 40 | 40 |
| Lactobionic acid, | 40 | 40 | 40 | 40 |
| mM | | | | |
| Glucose, mM | 10 | 10 | 10 | 10 |
| Adenosine, mM | 2 | 2 | 2 | 2 |
| Reduced glutathione, | 3 | 3 | 3 | 3 |
| mM | | | | |
| Dextran-40, mM | 1.5 | 1.5 | 1.5 | 1.5 |
| Potassium phosphate | 20 | 20 | 20 | 20 |
| monobasic, mM | | | | |
| Potassium carbonate, | 10 | 10 | 10 | 10 |
| mM | | | | |
| Potassium chloride, | 20 | 20 | 20 | 20 |
| mM | | | | |
| Sodium chloride, mM | 5 | 5 | 5 | 5 |
| Potassium hydroxide, | 20 | 20 | 20 | 20 |
| mM | | | | |
| Vitamin E, mM | 2 | 2 | 5 | 5 |
| 4-Hydroxyethyl- | 0 | 20 | 0 | 10 |
| piperazine | | | | |
| ethanesulfonic acid, | | | | |
| mM | | | | |

### Example 5

Measured the quality of the cell preservation solution in Examples 1~4 by benchmarking it with commercially available HypoThermosol® preservation solutions.

Specifically, cultured the experimental cell line of human colon cancer cells (HCT116 WT) in a Petri dish, after digestion with trypsin solution, collected the cell suspension, centrifuged at 1000 rpm for 5 min, discarded the supernatant, and resuspended the cells with the cell preservation solution samples from experimental Examples 1~4 and the HypoThermosol® preservation solution samples (comparison group), respectively, aliquoted at 2×10⁶/tube cell concentrations in sterile cryovials, put in a 4° C. refrigerator to refrigeration preserve for 5 days.

Then, took out some of the cryovials, centrifuged to collect the cells, aspirated the preservation solution, resuspended the cells with cell culture medium, and morphologically measured the percentage of viable cells with a Countess 3 automated cell counter, the results are shown in Table 2 below. It can be seen that the viability rate of cells preserved using the cell preservation solution samples from experimental Examples 1-4 is as high as 94%, which is comparable to or even higher than that of cells preserved using the control group. However, the cell preservation solutions of the embodiments of the present invention have simpler formula and lower cost than the comparison group formulation.

TABLE 2

| | % of cell viability |
|---|---|
| Sample | (morphology) |
| Example 1 | 94.7 |
| Example 2 | 95.3 |
| Example 3 | 96.2 |
| Example 4 | 96.6 |
| Comparison Group | 94.4 |

On the other hand, transferred cells from cell preservation solution samples from experimental Examples 1~4 and HypoThermosol® preservation solution sample (comparison group) refrigerated at 4° C. for 5 days to a 96-well dish for resuscitating culture at a volume of 4,000 cells/well. After 1 day, measured cell metabolic activity with ALAMAR BLUE™ cell viability reagent, and recorded cell proliferation activity simultaneously with Sartorius IncuCyte Real-Time Live-Cell Imaging Analyzer. Subsequently, removed the ALAMAR BLUE™ cell viability test solution, re-added fresh culture medium, resuscitating cultured, and repeated the ALAMAR BLUE™ cell viability test after 3 days, and recorded the cell proliferation activity simultaneously with the Sartorius IncuCyte Real-Time Live-Cell Imaging Analyzer. Then, removed the ALAMAR BLUE™ cell viability test solution, re-added the fresh culture medium, resuscitating cultured, and after 5 days, repeated the ALAMAR BLUE™ cell viability test reagent experiment to measure cell metabolic activity, and recorded the cell proliferation activity simultaneously with the Sartorius IncuCyte Real-Time Live-Cell Imaging Analyzer.

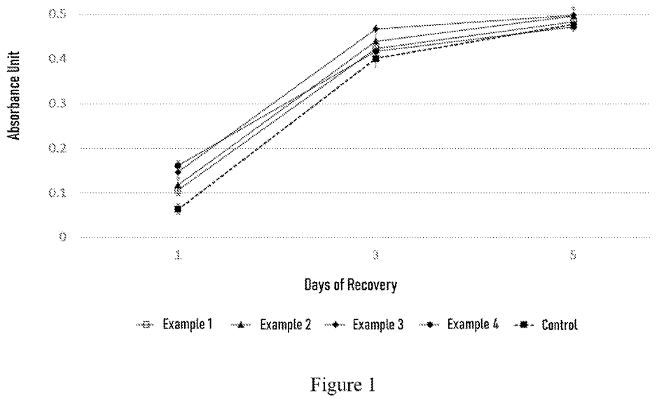

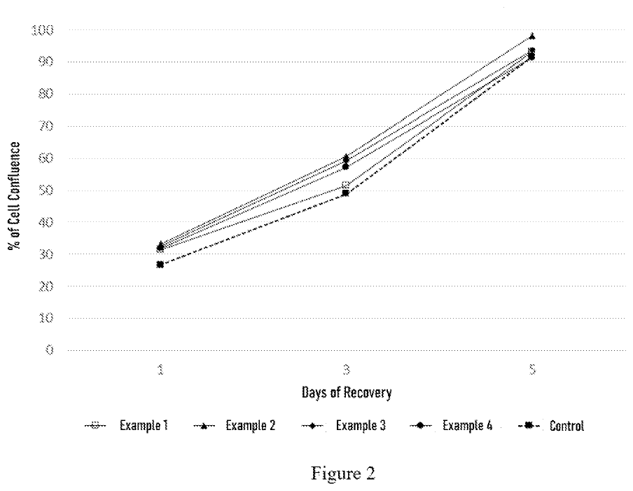

The data of cell metabolic activity at 1 day, 3 days, and 5 days of recovery from the above ALAMAR BLUE™ cell viability reagent measurement experiment are shown in FIG. 1, and the data of the cell proliferation activity experiments recorded by the Sartorius™ IncuCyte Real-Time Live Cell Imaging Analyzer are shown in FIG. 2. Refer to FIG. 1, the metabolic function of the cells preserved with the cell preservation solution samples from Example 1-4 was effectively restored, and the number of absorbance units increased with the number of days of recovery. As shown in FIG. 2, the cells preserved with the cell preservation solution samples from Examples 1-4 had normal growth, differentiation, and proliferation functions, indicating that the refrigerated cells were effectively recovered and had a higher degree of cell confluency with the increase of recovery days. And it can be seen that the performance of the cell preservation solutions of the embodiment of the present invention can be comparable to or better than that of the comparison group.

### Examples 6-9

Following the compositions and concentrations of the cell preservation solutions corresponding to the experimental Examples 6-9 shown in Table 3 below, individually weighed Sucrose, mannitol, lactobionic acid, glucose, adenosine, reduced glutathione, dextran-40, potassium dihydrogen phosphate, potassium carbonate, potassium chloride, sodium chloride, potassium hydroxide, 4-(2-hydroxyethyl)piperazine-1-ethanesulfonic acid (HEPES), dimethyl sulfoxide. Used purified water to prepare 4 kinds of mixed solutions, titrated each with sodium hydroxide to pH 7.6, and filtered through a 0.2 μm sterile filter membrane, and the filtered filtrates were the 4 kinds of cell preservation solutions in experimental Examples 6-9.

TABLE 3

| | Concentration | | | |
|---|---|---|---|---|
| | Example | Example | Example | Example |
| Ingredient | 6 | 7 | 8 | 9 |
| Sucrose, mM | 40 | 40 | 40 | 40 |
| Mannitol, mM | 40 | 40 | 40 | 40 |
| Lactobionic acid, mM | 40 | 40 | 40 | 40 |
| Glucose, mM | 10 | 10 | 10 | 10 |
| Adenosine, mM | 2 | 2 | 2 | 2 |
| Reduced glutathione, | 3 | 3 | 3 | 3 |
| mM | | | | |
| Dextran-40, mM | 1.5 | 1.5 | 1.5 | 1.5 |
| Potassium phosphate | 20 | 20 | 20 | 20 |
| monobasic, mM | | | | |
| Potassium carbonate, | 10 | 10 | 10 | 10 |
| mM | | | | |
| Potassium chloride, | 20 | 20 | 20 | 20 |
| mM | | | | |
| Sodium chloride, mM | 5 | 5 | 5 | 5 |
| Potassium hydroxide, | 20 | 20 | 20 | 20 |
| mM | | | | |
| 4-Hydroxyethyl- | 0 | 20 | 0 | 10 |
| piperazine | | | | |
| ethanesulfonic acid, | | | | |
| mM | | | | |
| Dimethyl sulfoxide, | 5 | 5 | 10 | 10 |
| % | | | | |

### Example 10

Measured the quality of the cell preservation solutions in Examples 6-9 by benchmarking it with commercially available CryoStor® CS5 and CryoStor® CS10 cell cryopreservation solutions.

Specifically, cultured the experimental cell line HCT116 WT in a Petri dish, after digestion with trypsin solution, collected the cell suspension, centrifuged at 1000 rpm for 5 min, discarded the supernatant, and resuspended cells with the cell preservation solution samples from experimental Examples 6-9 and the CryoStor® CS5 cell cryopreservation solution samples (comparison group 1) and CryoStor® CS10 cell cryopreservation solution samples (comparison group 2), respectively, aliquoted at a cell concentration of 2×10⁶/tube in sterile cryopreservation tubes, placed at −80° C. temperature condition at a cooling rate of approximately 1° C./min, and moved to liquid nitrogen (−196° C.) overnight for freezing preservation.

After 2 months of storage, took out the cryopreservation tube, quickly rewarmed at 37° C. condition, stopped rewarming when the ice crystals were about to completely thaw, after centrifugation collection, aspirated the cell preservation solutions and cryopreservation solutions, resuspended the cells with cell culture medium, and used the Countess 3 Automated Cell Counter to measure the percentage of viable cells morphologically. The results are shown in Table 4 below, and it can be seen that the survival rate of cells preserved using the cell preservation solution samples from Examples 6-9 is more than 95%, which is comparable to or even higher than that of cells preserved using control groups 1 and 2. However, the cell preservation solutions of the embodiment of the present invention have simpler formula and lower cost than the comparison group 1 and 2 formulations.

TABLE 4

| | % of cell viability |
|---|---|
| Sample | (morphology) |
| Example 6 | 95.7 |
| Example 7 | 97.2 |
| Example 8 | 96.9 |
| Example 9 | 95.7 |
| Comparison Group 1 | 94.9 |
| Comparison Group 2 | 95.6 |

On the other hand, transferred the cells from the cell preservation solution samples described above in Example 6-9 and the cells in comparison groups 1 and 2 cryopreserved for 2 months to a 96-well dish for resuscitating culture at a volume of 4000 cells/well, after 1 day, measured the metabolic activity of the cells with the ALAMAR BLUE™ Cell Viability Assay Reagent, and recorded the cell proliferation activity simultaneously with the Sartorius IncuCyte Real-Time Live Cell Imaging Analyzer. Subsequently, removed the ALAMAR BLUE™ cell viability test solution, re-added fresh culture medium, resuscitating cultured, and repeated the ALAMAR BLUE™ cell viability test after 3 days, and recorded the cell proliferation activity simultaneously with the Sartorius IncuCyte real-time live-cell imaging analyzer. Then, removed the ALAMAR BLUE™ cell viability test solution, re-added the fresh culture medium, resuscitating cultured, and after 5 days, repeated the ALAMAR BLUE™ cell viability test reagent experiment to measure cell metabolic activity, and recorded the cell proliferation activity simultaneously with the Sartorius IncuCyte real-time live-cell imaging analyzer.

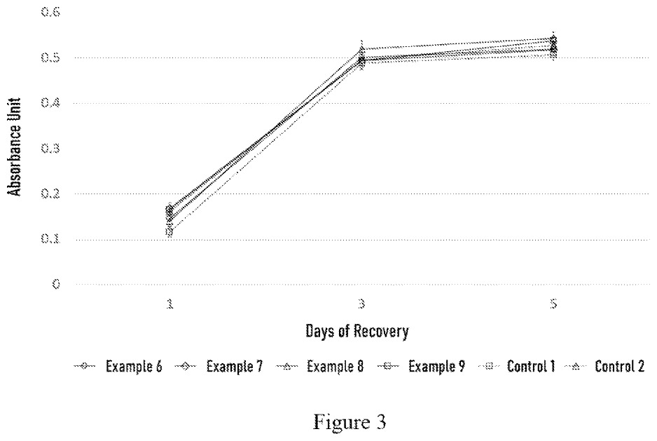

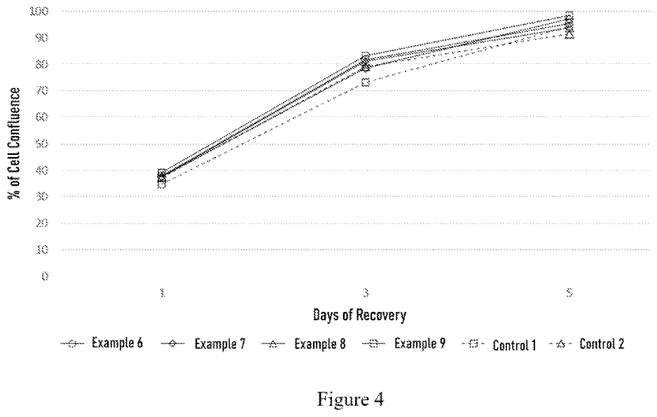

The data of cell metabolic activity at 1 day, 3 days, and 5 days of recovery from the ALAMAR BLUE™ cell viability reagent measurement experiment are shown in FIG. 3, and the data of cell proliferation activity at 1 day, 3 days, and 5 days of recovery recorded by the Sartorius IncuCyte Real-Time Live-Cell Imaging Analyzer are shown in FIG. 4. See FIG. 3, cells preserved with a sample of cell preservation solution from Example 6-9 have an effective recovery of cellular metabolism, with a higher number of resorption units as the number of days of recovery increases. As shown in FIG. 4, the cells preserved with the cell preservation solution samples from Example 6-9 had normal growth, differentiation, and proliferation, indicating that the frozen cells were effectively recovered and had higher cell confluency with the increase of recovery days. And it can be seen that the performance of the cell preservation solutions of the embodiments of the present invention can be comparable to or even better than that of the comparison groups 1 and 2.

The various embodiments described above and shown in the accompanying drawings are only for illustrative purposes of the present invention and are not all of the present invention. Within the scope of the basic technical idea of the present invention, any form of alteration made by a person skilled in the relevant technical field for the present invention is within the scope of protection of the present invention.

1. A cell preservation solution, comprising:
20 mM~50 mM sucrose;
20 mM~50 mM mannitol;
10 mM~50 mM lactobionic acid;
5 mM~20 mM glucose;
1 mM~3 mM adenosine;
1 mM~5 mM reduced glutathione;
1 mM~2 mM dextran-40;
10 mM~30 mM potassium dihydrogen phosphate;
10 mM~30 mM potassium carbonate;
10 mM~30 mM potassium chloride;
5 mM~30 mM sodium chloride;
10 mM~60 mM sodium hydroxide;
10 mM~60 mM potassium hydroxide;
0 mM~20 mM vitamin E;
0 mM~25 mM 4-hydroxyethylpiperazine ethanesulfonic acid (HEPES); and
0%~10% dimethyl sulfoxide.

2. The cell preservation solution according to claim 1, comprising 0.5 mM~20 mM vitamin E and 0% dimethyl sulfoxide.

3. The cell preservation solution according to claim 2, comprising 2 mM~5 mM vitamin E.

4. The cell preservation solution according to claim 1, comprising 0 mM~20 mM 4-hydroxyethylpiperazine ethanesulfonic acid (HEPES).

5. The cell preservation solution according to claim 1, comprising 0 mM vitamin E and >0% dimethyl sulfoxide.

6. The cell preservation solution according to claim 5, comprising 0 mM~20 mM 4-hydroxyethylpiperazine ethanesulfonic acid (HEPES).

7. The cell preservation solution according to claim 5, comprising 5%~10% dimethyl sulfoxide.

8. The cell preservation solution according to claim 1, having a pH value in the range of 7.0~8.0.

9. The cell preservation solution according to claim 1, having an osmotic pressure of 300~400 milliosmoles.

10. The cell preservation solution according to claim 1, comprising water.

11. A method of cell preservation, using a cell preservation solution according to claim 1 to preserve cells.

12. The cell preservation method of claim 11, preserving cell at 2° C.~8° C. or −80° C.~−196° C.

13. The cell preservation method of claim 11, wherein the cells comprise one or more of stem cells, immune cells, and tumor cells.

14. The cell preservation method of claim 11, wherein the cells comprises human colon cancer cells.

15. The cell preservation method of claim 11, wherein the cell preservation solution comprises 2 mM~5 mM vitamin E.

16. The cell preservation method of claim 11, wherein the cell preservation solution comprises 0 mM~20 mM 4-hydroxyethylpiperazine ethanesulfonic acid (HEPES).

17. The cell preservation method of claim 11, wherein the cell preservation solution comprises 0 mM vitamin E and >0% dimethyl sulfoxide.

18. The cell preservation method of claim 11, wherein the cell preservation solution comprises 5%~10% dimethyl sulfoxide.

19. The cell preservation method of claim 11, wherein the cell preservation solution has a pH value in the range of 7.0~8.0.

20. The cell preservation method of claim 11, wherein the cell preservation solution has an osmotic pressure of 300~400 milliosmoles.
